(19) United States
(12) Patent Application Publication
(10) Pub. No.: US 2026/0188527 A1
(43) Pub. Date: Jul. 2, 2026

(54) BIOLOGICAL INFORMATION COLLECTING APPARATUS, INFORMATION PROCESSING APPARATUS, INFORMATION PROCESSING METHOD, AND RECORDING MEDIUM

(71) Applicant: CANON MEDICAL SYSTEMS CORPORATION, Tochigi (JP)

(72) Inventors: Taiga YOSHINO, Utsunomiya (JP); Yuto TAKAHASHI, Otawara (JP); Koki KAWAI, Utsunomiya (JP); Kyomi UCHINO, Utsunomiya (JP); Junichi TAKAZAWA, Nasushiobara (JP)

(73) Assignee: CANON MEDICAL SYSTEMS CORPORATION, Tochigi (JP)

(21) Appl. No.: 19/433,408

(22) Filed: Dec. 26, 2025

(30) Foreign Application Priority Data
Dec. 26, 2024 (JP) 2024-229840
Dec. 25, 2025 (JP) 2025-281710

(51) Int. Cl. G16H 70/60; G06T 11/26; G16H 50/70

(52) U.S. Cl. CPC G16H 70/60; G06T 11/26; G16H 50/70; G06T 2210/41

(57) ABSTRACT

A biological information collecting apparatus according to the present embodiments includes processing circuitry. The processing circuitry acquires information about the condition of a patient. The processing circuitry calculates indexes related to occurrence of a plurality of diseases, based on the acquired information. The processing circuitry causes a display to display the index related to occurrence of each of the diseases.

### CROSS-REFERENCE TO RELATED APPLICATIONS

This application is based upon and claims the benefit of priority from Japanese Patent Application No. 2024-229840, filed on Dec. 26, 2024; and Japanese Patent Application No. 2025-281710, filed on Dec. 25, 2025, the entire contents of which are incorporated herein by reference.

### FIELD

Embodiments described herein relate generally to a biological information collecting apparatus, an information processing apparatus, and an information processing method, and a recording medium.

### BACKGROUND

At the scene of emergency, fast decision-making and treatment are critical to save the life of a patient. For example, when a patient is seriously injured, the chance of saving the life of the patient decreases with time, hence it is important to perform differentiation for narrowing down a disease, based on the condition of the patient (vital signs and physical findings).

In recent years, while demand for emergency medical services has increased, a shortage of emergency physicians has emerged. As a result, personnel cannot be sufficiently assigned at the scene of emergency, which results in a possibility that an inexperienced physician (for example, a resident or a physician who is outside his/her field) performs differentiation. In recent years, due to the adjustment of physicians'working hours as a result of the work style reform, there is also a possibility that physicians cannot be sufficiently assigned. Currently, however, there is no means to assist differentiation. Therefore, not only assisting differentiation, but also reducing time required for differentiation are desired.

### BRIEF DESCRIPTION OF THE DRAWINGS

FIG. 1 is a diagram illustrating an example of the configuration of an information processing system including an information processing apparatus according to the present embodiment;

FIG. 2 is a diagram illustrating an example of the configuration of the information processing apparatus according to the present embodiment;

FIG. 3 is a diagram illustrating an example of a workflow in emergency outpatient treatment;

FIG. 4 is a flowchart illustrating the procedure of processing performed by the information processing apparatus according to the present embodiment;

FIG. 5 is a diagram illustrating a display example displayed by a display of the information processing apparatus according to the present embodiment;

FIG. 6 is a diagram illustrating the occurrence probabilities of a plurality of diseases displayed in a display area of a screen in FIG. 5;

FIG. 7 is a diagram illustrating a display example in which criteria for each disease are displayed in the display area of the screen in FIG. 5;

FIG. 8 is a diagram illustrating a display example in which score items of a criterion are displayed in graph form in the display area of the screen in FIG. 5;

FIG. 9 is a diagram illustrating a display example in which score items of a criterion are displayed in graph form in the display area of the screen in FIG. 5;

FIG. 10 is a diagram illustrating the occurrence probabilities of a plurality of diseases displayed in the display area of the screen in FIG. 5 and illustrating a display example in which the number of diseases displayed in the graph of occurrence probability is large;

FIG. 11 is a diagram illustrating the occurrence probabilities of a plurality of diseases displayed in the display area of the screen in FIG. 5 and illustrating a display example in which the number of diseases displayed in the graph of occurrence probability is large;

FIG. 12 is a diagram of a first modification illustrating an example of a workflow in emergency outpatient treatment and illustrating a display example in which images are used;

FIG. 13 is a diagram of the first modification illustrating the occurrence probabilities of a plurality of diseases displayed in the display area of the screen in FIG. 5 and illustrating a display example in which images are used;

FIG. 14 is a diagram of the first modification illustrating a display example in which criteria for each disease are displayed in the display area of the screen in FIG. 5 and illustrating a display example in which images are used;

FIG. 15 is a diagram of a second modification illustrating a case in which occurrence probabilities are identical;

FIG. 16 is a flowchart of the second modification illustrating a procedure of processing performed when occurrence probabilities are identical;

FIG. 17 is a diagram of the second modification illustrating processing performed when the conditions of criteria are satisfied and the difference between a threshold and an actual measured value is large;

FIG. 18 is a diagram of the second modification illustrating processing performed when the conditions of the criteria are not satisfied and the difference between a threshold and an actual measured value is small;

FIG. 19 is a diagram of a third modification illustrating a preferential display of parameters based on a disease;

FIG. 20 is a diagram illustrating a display example in a fourth modification;

FIG. 21 is a diagram illustrating a display example in a fifth modification;

FIG. 22 is a diagram illustrating another display example in the fifth modification;

FIG. 23 is a diagram illustrating an example of the configuration of an information processing system including a biological information collecting apparatus; and

FIG. 24 is a diagram illustrating an example of the configuration of the biological information collecting apparatus.

### DETAILED DESCRIPTION

A biological information collecting apparatus according to the present embodiments includes processing circuitry. The processing circuitry acquires information about the condition of a patient. The processing circuitry calculates indexes related to occurrence of a plurality of diseases, based on the acquired information. The processing circuitry causes a display to display the index related to occurrence of each of the diseases.

Hereinafter, embodiments of the biological information collecting apparatus, an information processing apparatus, an information processing method, and a recording medium will be described in detail with reference to the drawings. An information processing system including the information processing apparatus is described below as an example.

### First Embodiment

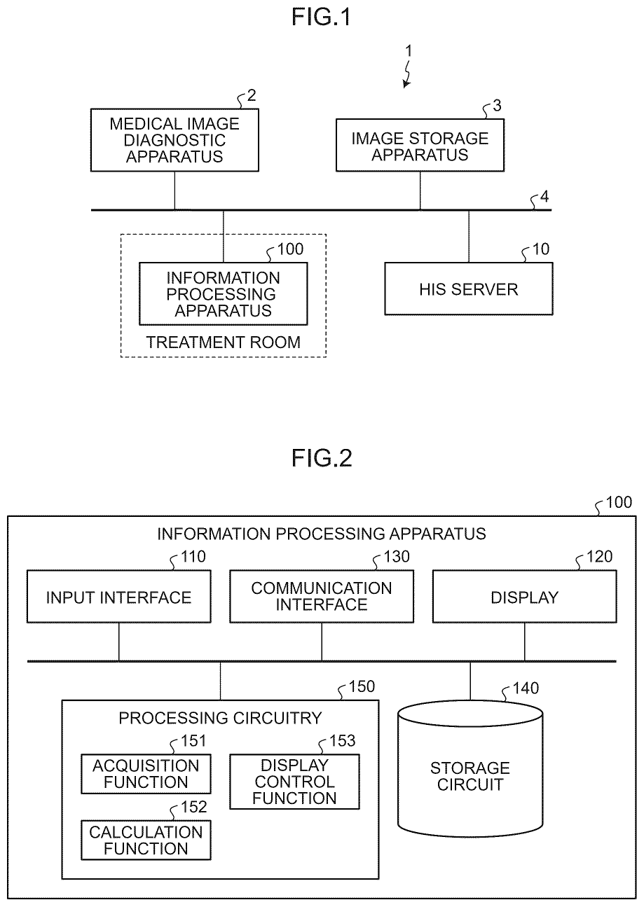

FIG. 1 is a diagram illustrating an example of the configuration of an information processing system 1 including an information processing apparatus 100 according to the present embodiment. The information processing system 1 illustrated in FIG. 1 includes the information processing apparatus 100, a medical image diagnostic apparatus 2, an image storage apparatus 3, and a hospital information system (HIS) server 10. The information processing apparatus 100 is connected to the medical image diagnostic apparatus 2, the image storage apparatus 3, and the HIS server 10 by a network 4, such as an in-hospital local area network (LAN) installed in a hospital. Here, the devices can communicate with each other directly or indirectly. For example, when a picture archiving and communication system (PACS) is installed in the information processing system 1, the devices transmit and receive medical images and other data to and from each other in accordance with the digital imaging and communications in medicine (DICOM) standard.

The HIS server 10 manages information generated in the hospital. The information generated in the hospital includes, for example, patient information and test order information. The patient information includes basic information, medical information, and test implementation information about a patient. The basic information includes patient ID, name, the date of birth, gender, blood type, height, and weight. For the patient ID, identification information is set to uniquely identify the patient. The medical information about the patient includes information, such as numerical values (measured values) and medical records, and information indicating the record date and time of the information. Examples of the medical information about the patient include drug prescriptions made by a physician, care records made by a nurse, tests in a medical test department, and dietary arrangements during hospitalization. For example, prescriptions are recorded in an electronic medical record by a physician, and care records are recorded in the electronic medical record by a nurse. The test implementation information includes information about tests implemented in the past and the results of the tests and information indicating the implementation dates of the tests.

Examples of the medical image diagnostic apparatus 2 include an X-ray diagnostic device, an X-ray computed tomography (CT) device, a magnetic resonance imaging (MRI) device, an ultrasonic diagnostic device, and a single photon emission computed tomography (SPECT) device. Further examples of the medical image diagnostic apparatus 2 include a positron emission computed tomography (PET) device, a SPECT-CT device in which a SPECT device and an X-ray CT device are combined, a PET-CT device in which a PET device and an X-ray CT device are combined, and a group of these devices. The medical image diagnostic apparatus 2 can generate a two-dimensional medical image, a three-dimensional medical image (volume data), time-series two-dimensional medical images, and time-series three-dimensional medical images.

Here, the medical image diagnostic apparatus 2 collects medical images by imaging a subject, based on the patient information and the test order information from the HIS server 10. For example, an X-ray CT device serving as the medical image diagnostic apparatus 2 causes an X-ray tube and an X-ray detector to circle around a subject to which a contrast agent is administered, and detects X-rays transmitted through the subject to collect projection data. Based on the collected projection data, the X-ray CT device then generates a two-dimensional CT image, a three-dimensional CT image (volume data), time-series two-dimensional CT images, or time-series three-dimensional CT images. Alternatively, the X-ray CT device generates a plurality of two-dimensional CT images along a predetermined direction, based on the collected projection data. For example, the X-ray CT device generates a plurality of two-dimensional CT images of axial cross sections taken along a body axis.

The medical image diagnostic apparatus 2 transmits the generated medical images to the image storage apparatus 3. Note that, when transmitting the medical images to the image storage apparatus 3, the medical image diagnostic apparatus 2 transmits supplementary information, such as patient ID to identify a patient, examination ID to identify a test, device ID to identify the medical image diagnostic apparatus 2, and series ID to identify single imaging performed by the medical image diagnostic apparatus 2.

The image storage apparatus 3 is a database configured to store medical images. Specifically, the image storage apparatus 3 includes a storage circuit and stores medical images transmitted from the medical image diagnostic apparatus 2 by means of storing the medical images in the storage circuit. Examples of the storage circuit of the image storage apparatus 3 include semiconductor memory elements, such as random access memory (RAM) and flash memory, and storage devices, such as a hard disk and an optical disk. Note that the medical images are associated with the patient ID, the test ID, the device ID, the series ID, and other IDs and stored in the image storage apparatus 3. Therefore, the information processing apparatus 100 can retrieve necessary medical images from the image storage apparatus 3 by performing a search using the patient ID, the test ID, the device ID, the series ID, and other IDs.

The information processing apparatus 100 is a device configured to perform processing of various types of medical information, and examples of the information processing apparatus 100 include a workstation, a personal computer (PC), a tablet PC, a personal digital assistant or personal data assistant (PDA), and a cell phone such as a smartphone. The information processing apparatus 100 is capable of applying various types of processing to medical information acquired from the HIS server 10, the medical image diagnostic apparatus 2, the image storage apparatus 3, and other devices. In the present embodiment, the information processing apparatus 100 is provided in a treatment room in which treatment is given to emergency patients, and the information processing apparatus 100 mainly performs processing by using patient information obtained in the treatment room.

FIG. 2 is a diagram illustrating an example of the configuration of the information processing apparatus 100 according to the present embodiment. As illustrated in FIG. 2, the information processing apparatus 100 includes an input interface 110, a communication interface 130, a display 120, a storage circuit 140, and processing circuitry 150.

The input interface 110 is realized, for example, with a trackball, a switch button, a mouse, a keyboard, a touch pad configured to allow input operation to be performed with a touch of an operation face, a touch screen in which a display screen and the touch pad are combined, a non-contact input interface using an optical sensor, and a voice input interface. The input interface 110 receives inputs of various operations for the information processing apparatus 100 from a physician as a user and transfers instruction and setting information received from the user to the processing circuitry 150.

The display 120 is a monitor referred to by the user. The display 120 displays images to the user under the control by the processing circuitry 150 or displays a graphical user interface (GUI) to receive various instructions, various settings, and the likes from the user via the input interface 110. The communication interface 130 is, for example, a network interface card (NIC) and communicates with other devices. The display 120 is an example of a display unit.

The storage circuit 140 is, for example, a semiconductor memory element such as RAM or a flash memory, or a storage device such as a hard disk or an optical disk. The storage circuit 140 stores various types of patient medical information to be used for processing and guideline information for calculating occurrence probability described later.

The processing circuitry 150 controls constituents of the information processing apparatus 100. For example, the processing circuitry 150 performs an acquisition function 151, a calculation function 152, and a display control function 153, as illustrated in FIG. 2. Here, for example, processing functions performed by the acquisition function 151, the calculation function 152, and the display control function 153, which are the constituents of the processing circuitry 150, are recorded in the storage circuit 140 in the form of a program executable by a computer. The processing circuitry 150 is a processor configured to read programs from the storage circuit 140 and execute the programs and thereby realize the functions respectively corresponding to the programs. In other words, by reading the programs, the processing circuitry 150 obtains the functions illustrated in the processing circuitry 150 in FIG. 2. Note that the acquisition function 151, the calculation function 152, and the display control function 153 are examples of “an acquisition unit”, “a calculation unit”, and “a display control unit”, respectively.

The term “processor” used in the above description means, for example, a circuit, such as a central processing unit (CPU), a graphics processing unit (GPU), or an application specific integrated circuit (ASIC). In addition, the term “processor” means a circuit such as a programmable logic device. Examples of the programmable logic device include a simple programmable logic device (SPLD) and a complex programmable logic device (CPLD). Further examples of the programmable logic device include a field programmable gate array (FPGA). When the processor is, for example, a CPU, the processor reads and executes a program stored in the storage circuit 140 to realize a function. In contrast, when the processor is, for example, an ASIC, a program is incorporated directly into the circuit of the processor, instead of storing the program in the storage circuit 140. Note that each of the processors in the present embodiment is not limited to a processor configured as a single circuit, but may be configured as a single processor including a combination of a plurality of independent circuits to realize the functions thereof. Alternatively, the constituents installed in FIG. 2 may be integrated into a single processor to realize the functions thereof.

At the scene of emergency, fast decision-making and treatment are critical to save the life of a patient. For example, when a patient is seriously injured, the chance of saving the life of the patient decreases with time, hence it is important to perform differentiation for narrowing down a disease, based on the condition of the patient (vital signs and physical findings). In recent years, while demand for emergency medical services has increased, a shortage of emergency physicians has emerged. As a result, personnel cannot be sufficiently assigned at the scene of emergency, which results in a possibility that an inexperienced physician (for example, a resident or a physician who is outside his/her field) performs differentiation. In recent years, due to the adjustment of physicians'working hours as a result of the work style reform, there is also a possibility that physicians cannot be sufficiently assigned. Currently, however, there is no means to assist differentiation. Therefore, not only assisting differentiation, but also reducing time required for differentiation are desired.

Therefore, to enhance the efficiency of differentiation, the information processing apparatus 100 according to the present embodiment performs the following processing. First, in the information processing apparatus 100 according to the present embodiment, the acquisition function 151 acquires information about the condition of a patient. The calculation function 152 calculates indexes related to occurrence of a plurality of diseases, based on the acquired information. The display control function 153 causes the display 120 to display the index related to occurrence of each of the diseases.

Here, the index related to occurrence is important for physicians to perform differentiation for narrowing down a disease. The index related to occurrence is expressed by, for example, a numerical value or a symbol to represent the possibility of a disease having occurred, and is used as a criterion or a standard to facilitate understanding of the probability of a disease having occurred.

For example, when the possibility of a disease having occurred is expressed by a numerical value, an occurrence probability or a five-point scale is used as the index related to occurrence. For example, the occurrence probability is expressed within a range of “0%” to “100%”, and “100%” means the highest possibility of the occurrence of a disease. For example, the five-point scale is expressed within a range of “1” to “5”, “5” meaning the highest possibility of the occurrence of a disease.

For example, when the possibility of a disease having occurred is expressed by a symbol, symbols such as “○”, “Δ”, and “×” or a five-point scale are used as the index related to occurrence. For example, in the case of using symbols such as “○”, “Δ”, and “×”, “×” means the highest possibility of the occurrence of a disease, while “○” means the lowest possibility of the occurrence of the disease. For example, the five-point scale is expressed by using “A”, “B”, “C”, “D”, and “E”, and “E” means the highest possibility of the occurrence of a disease, while “A” means the lowest possibility of the occurrence of the disease.

An example in which occurrence probability is used as the index related to occurrence will be described below.

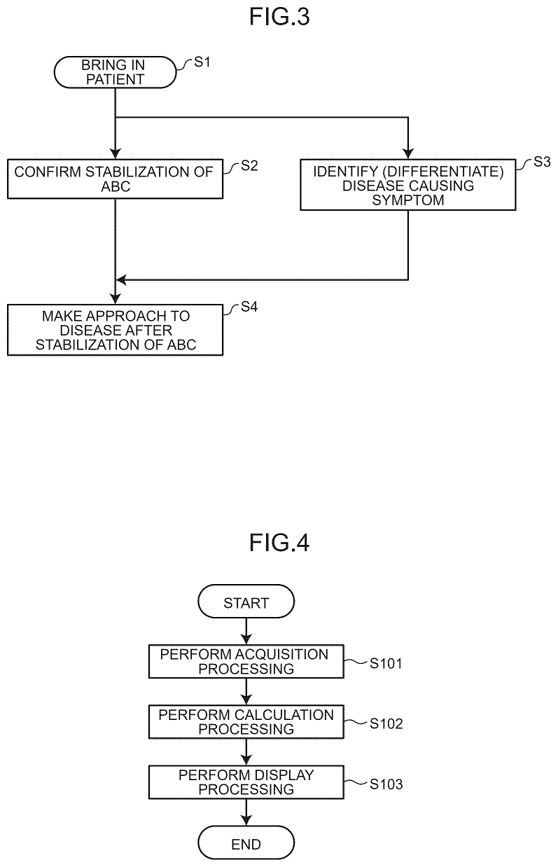

FIG. 3 is a diagram illustrating an example of a workflow in emergency outpatient treatment. As described above, at the scene of emergency, fast decision-making and treatment are critical to save the life of a patient. Therefore, for example, steps illustrated in FIG. 3 are performed in emergency outpatient treatment.

As illustrated in FIG. 3, first, a patient is brought in (step S1). Here, when the patient is brought in, the ABC approach is used to avoid a danger of life and the stabilization of the ABC is confirmed to stabilize vital signs (step S2). Here, the ABC approach is a technique used to prevent a patient from taking a sudden turn for the worse and summarizes the order of priority of observation and resuscitation in accordance with the oxygen flow of a living body (Airway, Breathing, and Circulation). As important workflow performed simultaneously with step S2, identification (differentiation) of a disease causing symptoms is performed (step S3). Then, after the stabilization of ABC, treatment is performed as an approach to the disease (step S4). Note that step S2 and step S3 are repeated until ABC is stabilized.

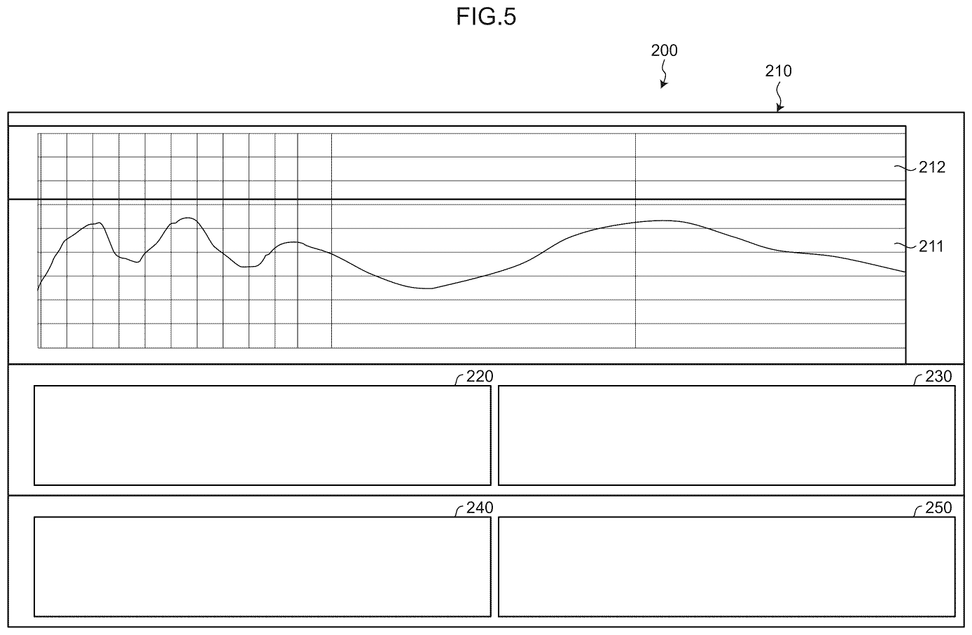

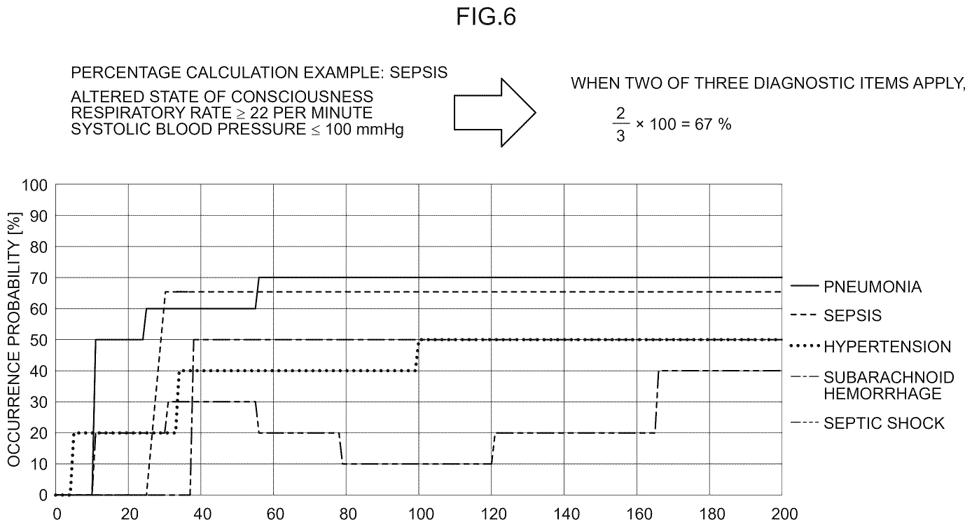

In the information processing apparatus 100 according to the present embodiment, differentiation is assisted at step S2. FIG. 4 is a flowchart illustrating the procedure of processing by the information processing apparatus 100 according to the present embodiment. FIG. 5 is a diagram illustrating a display example of a screen 200 displayed by the display 120 of the information processing apparatus 100 according to the present embodiment. FIG. 6 is a diagram illustrating the occurrence probabilities of a plurality of diseases displayed in a display area 212 of the screen 200 in FIG. 5.

Step S101 in FIG. 4 is a step at which the processing circuitry 150 reads out a program corresponding to the acquisition function 151 from the storage circuit 140 and executes the program. At step S101, the acquisition function 151 acquires information about the condition of a patient. For example, the HIS server 10 manages: information transmitted from a terminal belonging to an ambulance crew as patient information of a patient at the time of ambulance transportation of the patient; and patient information of the patient, the patient information being generated in a hospital after the ambulance transportation, and the acquisition function 151 acquires the patient information of the patient, information about the condition of the patient (consciousness and vitals), and the like from the HIS server 10. Furthermore, the acquisition function 151 sequentially acquires information about the patient that is acquired in a treatment room in which the patient is brought.

Step S102 in FIG. 4 is a step at which the processing circuitry 150 reads out a program corresponding to the calculation function 152 from the storage circuit 140 and executes the program. At step S102, the calculation function 152 calculates the occurrence probabilities of a plurality of diseases, based on the information acquired by the acquisition function 151. For example, the calculation function 152 calculates the occurrence probabilities [%] of the diseases as possible diseases, based on the information acquired by the acquisition function 151 and diagnostic items in guidelines. Note that the guidelines used for calculating the occurrence probabilities may be established by a public organization such as an academic society or may be set in a hospital.

Descriptions will be given by using sepsis as an example of a possible disease, for example. As illustrated in the upper part of FIG. 6, examples of a diagnostic item for “sepsis” include the altered state of consciousness, a respiratory rate, and a systolic blood pressure.

In the diagnostic item of the altered state of consciousness, the disturbance of consciousness of a patient is diagnosed by determining whether the Glasgow coma scale (GCS) is lower than 15 points. For example, when the GCS is 15 points or higher, there is a possibility that the disease is “sepsis”.

In the diagnostic item of the respiratory rate, whether the respiratory rate of a patient is 22 or more per minute is determined. For example, when the respiratory rate is 22 or more per minute, there is a possibility that the disease is “sepsis”.

In the diagnostic item of the systolic blood pressure, whether the systolic blood pressure of the patient is 100 mmHg or lower is determined. For example, when the systolic blood pressure is lower than 100 mmHg, there is a possibility that the disease is “sepsis”.

Here, as illustrated in the upper part of FIG. 6, when two out of the three diagnostic items for “sepsis” apply, the calculation function 152 calculates the occurrence probability of sepsis at 67[%] by (⅔)×100=67.

Step S103 in FIG. 4 is a step at which the processing circuitry 150 reads out a program corresponding to the display control function 153 from the storage circuit 140 and executes the program. At step S103, the display control function 153 causes the display 120 to display the occurrence probability of each of the diseases, the occurrence probability being calculated by the calculation function 152.

For example, the display control function 153 causes the display 120 to display the screen 200 illustrated in FIG. 5. In the example illustrated in FIG. 5, display areas 210, 220, 230, 240, and 250 are displayed on the screen 200.

For example, in the display areas 220, 230, 240, and 250, basic information about a patient (patient ID, name, date of birth, gender, blood type, height, and weight), medical information about the patient (information, such as numerical values and measured values and medical records, and information indicating the record date and time of the information), and test implementation information about the patient (information about tests and the results of the tests and information indicating implementation dates of the tests), and medical images obtained by the tests are displayed as patient information of the patient.

For example, the display area 210 is an area in which data are displayed on a time-series basis, and includes display areas 211 and 212. In the display area 211, vital signs such as a heart rate are displayed as information on the condition of the patient, on a time-series basis. Note that plural types of vital signs, for example, a heart rate and a blood pressure may be displayed in the display area 211. The vital signs displayed in the display area 211 can be changed by input by a physician. In the display area 212, the occurrence probability of each of the diseases is displayed as information on the condition of the patient, on a time-series basis.

The data displayed in the display area 212 are sequentially updated. Specifically, the acquisition function 151 sequentially acquires information on the condition of the

patient (consciousness and vital signs), and the calculation function 152 updates the occurrence probabilities of the diseases, based on the sequentially acquired information. At this time, the display control function 153 sequentially updates vital signs to be displayed by the display 120 and also sequentially updates the occurrence probability of each of the diseases to be displayed by the display 120. For example, in the example illustrated in FIG. 5, the display control function 153 causes the display 120 to display the sequentially updated vital signs on a time-series basis and also causes the display 120 to display the sequentially updated occurrence probability of each of the diseases on a time-series basis.

The occurrence probability of each of the diseases displayed in the display area 212 of the screen 200 in FIG. 5 is illustrated in FIG. 6. As illustrated in the lower part of FIG. 6, the diseases displayed in the display area 212 are, for example, “pneumonia”, “sepsis”, “hypertension”, “subarachnoid hemorrhage”, and “septic shock”. In the display area 212, the horizontal axis represents a time axis [seconds] and the vertical axis represents occurrence probability [%].

For example, as illustrated in FIG. 6, in the display area 212, the latest occurrence probabilities of the diseases, “pneumonia”, “sepsis”, “hypertension” and “subarachnoid hemorrhage”, and “septic shock” are lower in this order. Higher occurrence probability leads to greater urgency. Note that “hypertension” and “subarachnoid hemorrhage” have the same occurrence probability.

In the information processing apparatus 100 according to the present embodiment, the display control function 153 causes the display 120 to display the sequentially updated occurrence probability of each of the diseases on a time-series basis and the diseases are displayed in descending order of occurrence probability, whereby differentiation can be assisted. Thus, a reduction in time required for differentiation can be expected. With the information processing apparatus 100 according to the present embodiment, the user can check the time-series display of vital signs, and thereby can check variations in occurrence probability in relation to variations in vital signs. Thus, the information processing apparatus 100 according to the present embodiment can enhance the efficiency of differentiation.

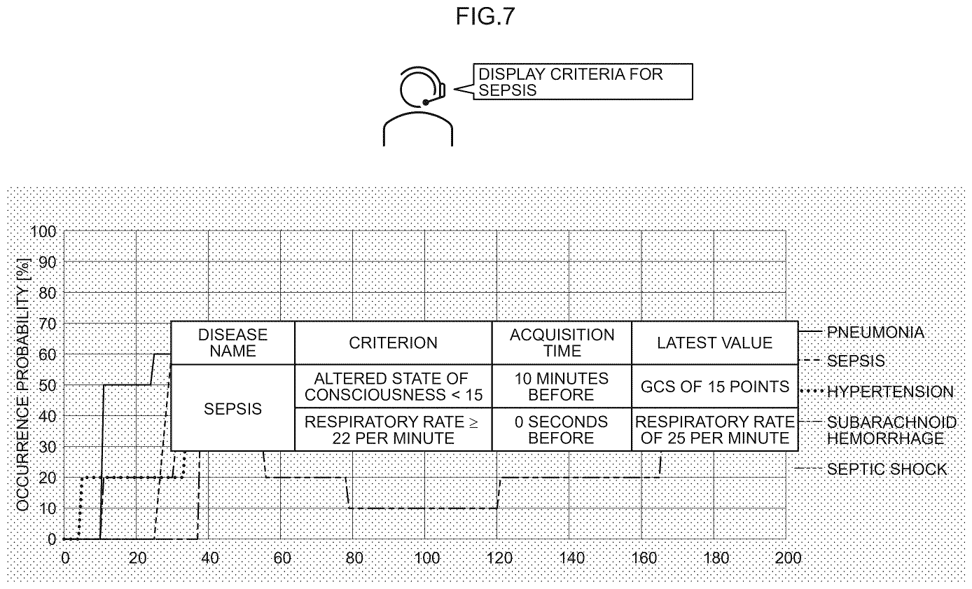

FIG. 7 is a diagram illustrating a display example in which criteria for each disease are displayed in the display area 212 of the screen 200 in FIG. 5.

The display control function 153 causes the display 120 to display information on criteria used to calculate the occurrence probability of a disease specified by user's input, out of a plurality of diseases. The information on criteria includes, for example, diagnostic items in guidelines used to determine a disease.

For example, it is assumed that, when the occurrence probability of each of the diseases is displayed in the display area 212 of the screen 200 in FIG. 5, the user provides an instruction to display criteria for sepsis.

In this case, the display control function 153 causes the criteria for sepsis to be displayed in the display area 212. Here, when the user provides an instruction “Display criteria for sepsis”, the display control function 153 causes the criteria for sepsis to be displayed in the display area 212 by voice input. Alternatively, when the user inputs the words “Display criteria for sepsis” by using the input interface 110, the display control function 153 causes the criteria for sepsis to be displayed in the display area 212 by text input.

For example, the display control function 153 causes the criteria for sepsis to be displayed in tabular form in the display area 212. Specifically, the display control function 153 associates the name of a disease, criteria for diagnostic items, the acquisition time of the diagnostic items, and the latest values of the diagnostic items with each other and causes them to be displayed in the display area 212.

In the example illustrated in FIG. 7, when two out of the three diagnostic items for “sepsis” apply, 67[%] is displayed as the occurrence probability of sepsis in the display area 212. For example, it is assumed that, in the diagnostic item of the altered state of consciousness, the latest value of GCS is 15 points as a disturbance of consciousness of the patient; in the diagnostic item of a respiratory rate, the latest value of the respiratory rate of the patient is 25 per minute; and in the diagnostic item of systolic blood pressure, the latest value of the systolic blood pressure of the patient is 100 mmHg or higher. Out of the three diagnostic items for “sepsis”, two diagnostic items, that is, the altered state of consciousness and the respiratory rate apply.

At this time, it is assumed that the user has provided an instruction “Display criteria for sepsis” to display the criteria for sepsis. In this case, in the example illustrated in FIG. 7, for the disease name “sepsis”, the criterion “the altered state of consciousness<15”, the acquisition time “10 minutes before”, and the latest value “GCS of 15 points” are displayed in the display area 212, and also the criterion “respiratory rate≥22/min”, the acquisition time “0 seconds before”, and the latest value “a respiratory rate of 25” are displayed therein.

Note that, in the example illustrated in FIG. 7, among diagnostic items for “sepsis”, the diagnostic items of the altered state of consciousness and the respiratory rate that indicate a possibility of “sepsis” are displayed in the display area 212, but the diagnostic item of the systolic blood pressure that does not indicate a possibility of “sepsis” may be displayed in the display area 212. Alternatively, all the diagnostic items of the altered state of consciousness, the respiratory rate, and the systolic blood pressure may be displayed in the display area 212, regardless of whether or not these diagnostic items indicate a possibility of “sepsis”.

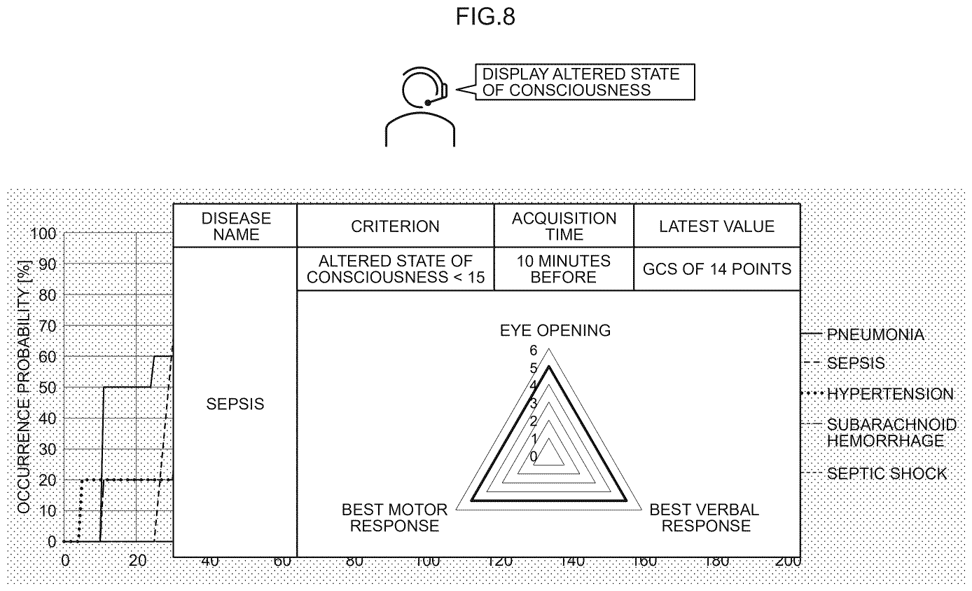

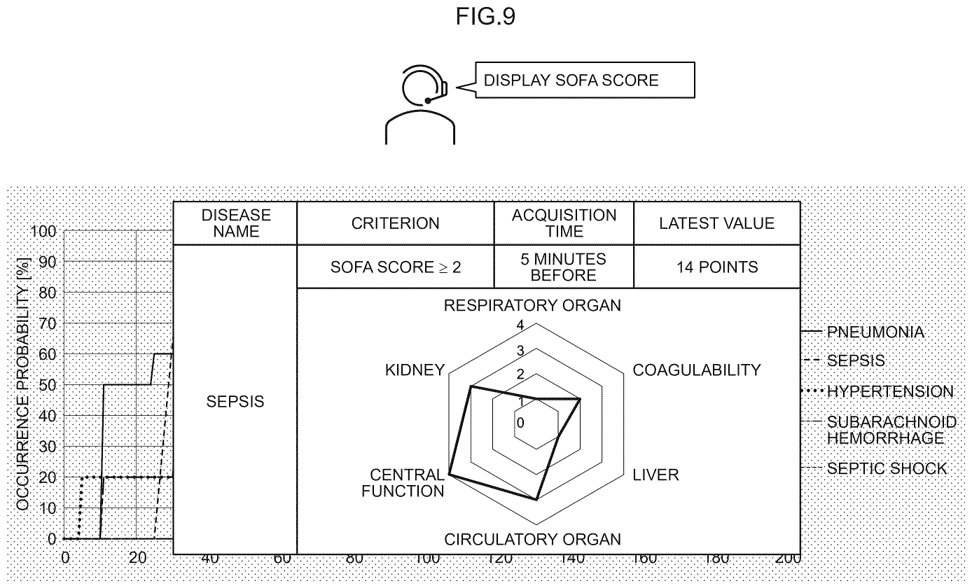

FIG. 8 and FIG. 9 are diagrams illustrating display examples in which score items of a criterion are displayed in graph form in the display area 212 of the screen 200 in FIG. 5.

The display control function 153 causes the display 120 to display the sequentially updated occurrence probability of each of the diseases in graph form on a time-series basis. Note that, in “correspondences between line types and symptoms (for example, a solid line indicates pneumonia)” displayed as the legends of the graph, the ranking of the occurrence probabilities may be reflected. That is, in the legends, the diseases may be displayed in descending order of the occurrence probability.

In the example illustrated in FIG. 8, it is assumed that, when the criteria for sepsis are displayed in the display area 212, the user provides, by voice input, an instruction “Display the altered state of consciousness” to display the diagnostic item of the altered state of consciousness of a patient as a disturbance of consciousness of the patient. Alternatively, it is assumed that, when the occurrence probability of each of the diseases is displayed in the display area 212 of the screen 200 in FIG. 5 and the user determines that there is a possibility of the patient having a disease “sepsis”, the user provides, by voice input, an instruction “Display the altered state of consciousness” to display the diagnostic item of the altered state of consciousness of the patient.

In this case, the display control function 153 causes the diagnostic item for sepsis, the altered state of consciousness, to be displayed in the display area 212. For example, the display control function 153 causes score items of a criterion as the diagnostic item for sepsis, the altered state of consciousness, to be displayed in graph form in the display area 212. Specifically, in the example illustrated in FIG. 8, the display control function 153 causes score items of a criterion as the diagnostic item for sepsis, the altered state of consciousness, to be displayed in graph form such as a radar chart in the display area 212.

In FIG. 8, for the disease name “sepsis”, the criterion “the altered state of consciousness: <15”, the acquisition time “10 minutes ago”, and the latest value “GCS of 15points” are displayed in the display area 212, and also the eye opening “5 points”, the best motor response “5 points” and the best verbal response “5 points” as the score items of the altered state of consciousness for sepsis are displayed therein in graph form. For example, the score of GCS includes information transmitted from a terminal belonging to an ambulance crew as patient information of a patient at the time of ambulance transportation of the patient and patient information of the patient that is generated in a hospital after the ambulance transportation.

In the example illustrated in FIG. 9, it is assumed that, when the criteria for sepsis are displayed in the display area 212, the user provides, by voice input, an instruction “Display Sequential Organ Failure Assessment (SOFA) score” to display the diagnostic item of the altered state of consciousness of a patient as a consciousness disorder of the patient. Alternatively, it is assumed that, when the occurrence probability of each of the diseases is displayed in the display area 212 of the screen 200 in FIG. 5 and the user determines that there is a possibility of a patient having the disease “sepsis”, the user provides, by voice input, an instruction “Display SOFA score” to display the diagnostic item of the altered state of consciousness of the patient. The SOFA score is determined by the calculation function 152, based on information acquired by the acquisition function 151 and SOFA score guidelines, for example.

In this case, the display control function 153 causes the diagnostic item for sepsis, the altered state of consciousness, to be displayed in the display area 212. For example, in the diagnostic item for sepsis, the altered state of consciousness, the display control function 153 causes score items of a criterion to be displayed as a SOFA score serving as an index of organ damage in graph form in the display area 212. Specifically, in the example illustrated in FIG. 9, the display control function 153 causes score items of a criterion to be displayed as a SOFA score serving as an index of organ damage in graph form such as a radar chart in the display area 212.

In FIG. 9, for the disease name “sepsis”, the criterion “the altered state of consciousness: <15”, the acquisition time “5 minutes ago”, and the latest value “GCS of 14 points” are displayed in the display area 212, and also “1 point” for respiratory organ, “3 points” for kidney, “4 points” for central function, “3 points” for circulatory organ, “1 point” for liver, and “2 points” for coagulability as the SOFA score items are displayed therein in graph form.

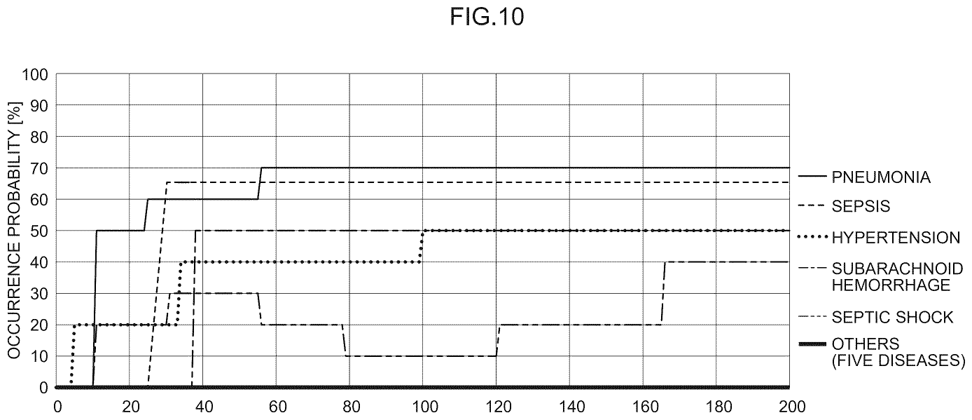

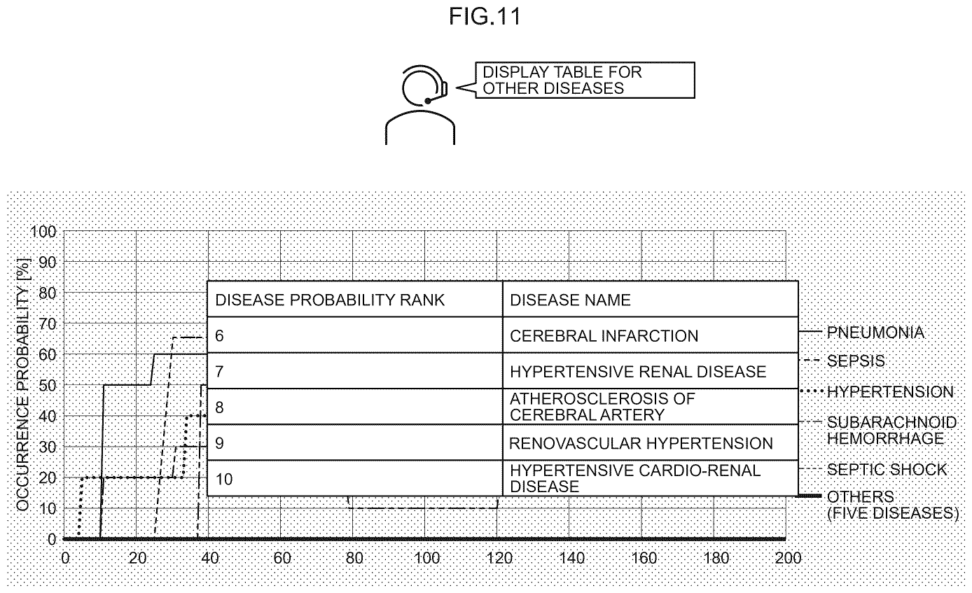

FIG. 10 and FIG. 11 are diagrams that illustrate the occurrence probability of each of the diseases displayed in the display area 212 of the screen 200 in FIG. 5 and illustrate a display example in which the number of diseases displayed in the graph of occurrence probability is large.

The display control function 153 causes the occurrence probability of a diseases satisfying a predetermined condition, out of a plurality of diseases, to be displayed. Here, the predetermined condition is a condition that occurrence probabilities ranking 1st to Nth are to be displayed. Note that the predetermined condition may be, for example, a condition that a disease with an occurrence probability of 10% or higher is to be displayed, or may be the number of diseases that can be displayed by the display 120 (in the present embodiment, the display area 212). The predetermined condition can be changed arbitrarily by an emergency physician.

In the example illustrated in FIG. 10, N=5. That is, in the example illustrated in FIG. 10, the occurrence probabilities of diseases having the latest occurrence probabilities ranking first to fifth are displayed in the display area 212. In the example illustrated in FIG. 5, in the display area 212, the time-series changes in the occurrence probability of each of the diseases, that is,

“pneumonia” ranking first, “sepsis” ranking second, “hypertension” and “subarachnoid hemorrhage” both ranking third, and “septic shock” ranking fifth, are displayed in graph form.

Here, diseases having occurrence probabilities ranking sixth or lower are collectively displayed using a single bold line at the lower part of the graph in the display area 212. In the example illustrated in FIG. 10, the diseases are collectively displayed using a single bold line at the lower part of the graph in the display area 212, and are displayed like “others (five diseases)” meaning that the diseases other than diseases “pneumonia”, “sepsis”, “hypertension”, “subarachnoid hemorrhage”, and “septic shock” and the number of the diseases not allowed to be displayed is five.

For example, on the request of the user, the display control function 153 causes the display 120 to display information about a disease not displayed.

For example, it is assumed that, when the occurrence probability of each of the diseases “pneumonia”, “sepsis”, “hypertension”, “subarachnoid hemorrhage”, and “septic shock” and also other diseases “others (five diseases)” is displayed in the display area 212 of the screen 200 in FIG. 5, the user provides an instruction to display the other diseases.

In the example illustrated in FIG. 11, it is assumed that, when the criteria for sepsis are displayed in the display area 212, the user provides, by voice input, an instruction “Display a table for other diseases” to display the other diseases. Alternatively, it is assumed that, when the occurrence probability of each of the diseases is displayed in the display area 212 of the screen 200 in FIG. 5, the user provides, by voice input, an instruction “Display a table for other diseases” to display the other diseases.

In this case, the display control function 153 causes the other diseases to be displayed in tabular form in the display area 212. Specifically, the display control function 153 associates the rank of disease probability of the diseases having occurrence probabilities ranking sixth or lower with the diseases with the occurrence probabilities ranking sixth or lower and causes them to be displayed in the display area 212.

In the example illustrated in FIG. 11, the disease names “cerebral infarction”, “hypertensive renal disease”, “atherosclerosis of cerebral artery”, “renovascular hypertension”, and “hypertensive cardio-renal disease” are displayed for the ranks of disease probability of “6”, “7”,

“8”, “9”, and “10”, respectively, in the display area 212.

As described above, in the information processing apparatus 100 according to the present embodiment, the display control function 153 causes the display 120 to display the sequentially updated occurrence probability of each of the diseases on a time-series basis, and the diseases are displayed in descending order of occurrence probability, whereby differentiation can be assisted. Thus, a reduction in time required for differentiation can be expected. With the information processing apparatus 100 according to the present embodiment, the user can check the time-series display of vital signs, and thereby can check variations in the occurrence probability in relation to variations in vital signs.

In addition, in the information processing apparatus 100 according to the present embodiment, on the request of the user, the display control function 153 causes the display 120 to display information about a disease not displayed, whereby the disease can be prevented from being overlooked and differentiation can be assisted.

Thus, the information processing apparatus 100 according to the present embodiment can enhance the efficiency of differentiation.

Note that time-series information about the occurrence probability and the like of each of the diseases, which is acquired in the present embodiment, may be associated with patient information of a patient and stored in the HIS server 10. Thus, information serving as grounds for differentiation performed at the time of ambulance transportation can be managed in a hospital.

In the present embodiment, the information processing apparatus 100 according to the present embodiment is provided in a treatment room in which emergency patients are treated. However, in the information processing apparatus 100, the input interface 110 and the display 120 may be provided in the treatment room, while other constituents, such as the storage circuit 140 and the processing circuitry 150, may be provided in other dispersed servers.

In the present embodiment, the information processing apparatus 100 causes the display 120 to display the sequentially updated occurrence probability of each of the diseases in time-series graph form, but is not limited to this and may cause the display 120 to display the latest occurrence probabilities of the diseases in tabular form.

### Other Embodiments

Besides the embodiment described above, various embodiments may be implemented.

### First Modification

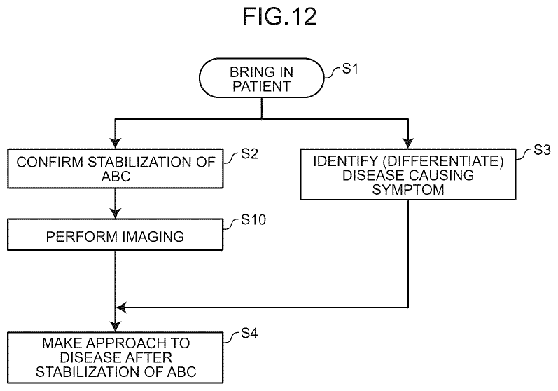

FIG. 12 is a diagram of a first modification illustrating an example of a workflow in emergency outpatient treatment and illustrating a display example in which images are used. In FIG. 12, first, a patient is brought in (step S1), then the stabilization of ABC is confirmed (step S2), and imaging is performed (step S10). At step S10, when an image is used, the presence or absence of findings such as a stroke is determined. As an important workflow performed simultaneously with step S2 and step S10, identification (differentiation) of a disease causing a symptom (step S3) is performed. Then, after the stabilization of ABC, treatment is performed as an approach to the disease (step S4). Note that steps S2 and S10 and step S3 are repeated until ABC is stabilized.

In the first modification, when image-based findings are present in at least one of a plurality of diseases, the display control function 153 causes the display 120 to display the presence of image-based findings for the disease.

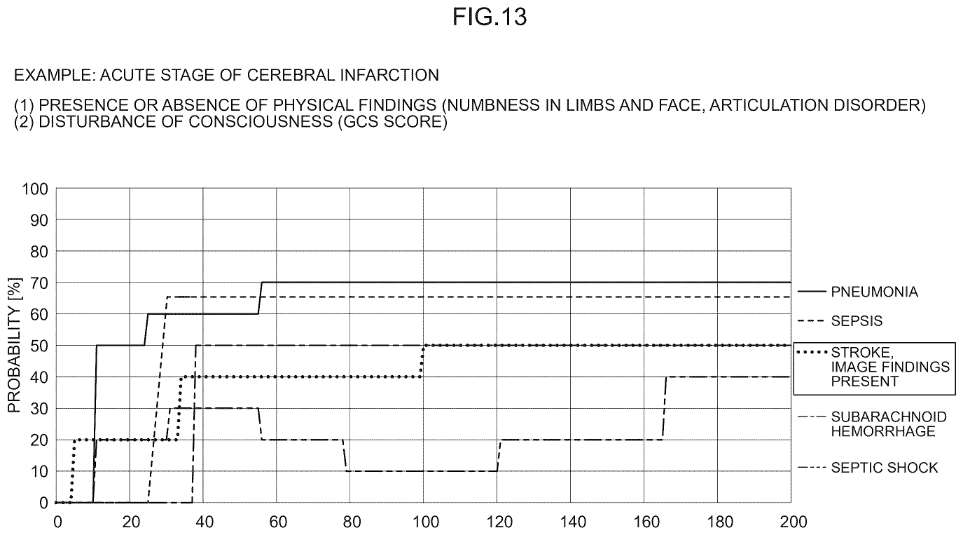

FIG. 13 is a diagram of the first modification illustrating the occurrence probability of each of the diseases displayed in the display area 212 of the screen 200 in FIG. 5 and illustrating a display example in which images are used.

In the example illustrated in FIG. 13, when an image is used, the image is not used for the calculation of disease probability, but the display control function 153 displays the presence of findings next to the name of a disease. For example, as illustrated in the upper part of FIG. 13, when the display control function 153 determines that findings are present as the acute stage of cerebral infarction, based on the presence or absence of physical findings (numbness in limbs and face, articulation disorder) and the disturbance of consciousness (GCS score) in the diagnosis items for “stroke”, the display control function 153 causes the disease “stroke” to be displayed in the display area 212 and furthermore causes the words “image findings present” to be displayed next to the disease “stroke” in the display area 212.

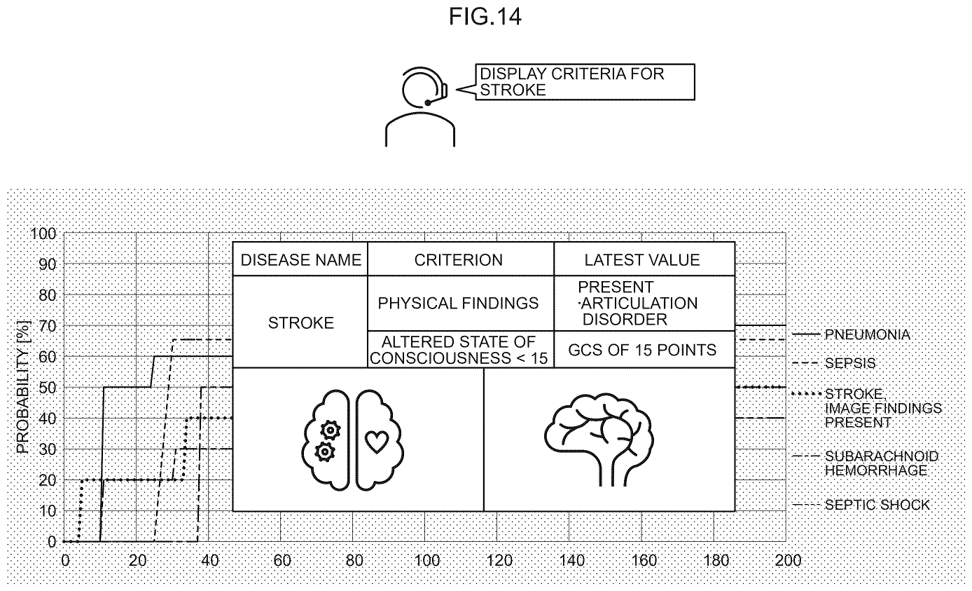

FIG. 14 is a diagram of the first modification illustrating a display example in which criteria for each disease are displayed in the display area 212 of the screen 200 in FIG. 5 and illustrating a display example in which images are used.

In the example illustrated in FIG. 14, it is assumed that, when the words “image findings present” are displayed in the display area 212 along with the disease “stroke”, the user provides, by voice input, an instruction “Display criteria for stroke” to display the criteria for stroke.

In this case, the display control function 153 causes the criteria for stroke to be displayed in the display area 212 along with images. In the example illustrated in FIG. 14, for the disease name “stroke”, the criterion “physical findings” and the latest value “present, articulation disorder” are displayed in the display area 212, and also the criterion “altered state of consciousness: <15” and the latest value “GCS of 15 points” are displayed therein. In addition, the images used for the disease “stroke” are displayed in the display area 212. For example, the images are acquired from the medical image diagnostic apparatus 2 or the image storage apparatus 3.

Thus, in the first modification, the display control function 153 causes the display 120 to display the presence of image-based findings for a disease, whereby the disease can be prevented from being overlooked.

### Second Modification

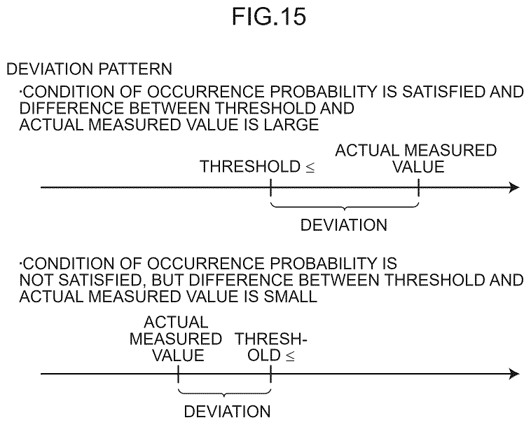

FIG. 15 is a diagram of a second modification illustrating a case in which occurrence probabilities are identical.

When ranking is determined for a plurality of diseases by occurrence probability, the display control function 153 adjusts the ranking, based on the difference between a threshold for a criterion used to calculate the occurrence probability and an actual measured value. For example, the display control function 153 adjusts the ranking of diseases having the same occurrence probability, based on the difference between a threshold for a criterion used to calculate the occurrence probabilities of the diseases and an actual measured value. Specifically, when a plurality of diseases has the same occurrence probability, the display control function 153 displays the diseases in consideration of the deviation between a threshold for a criterion used to calculate the occurrence probability and an actual measured value (the difference between the threshold and the actual measured value).

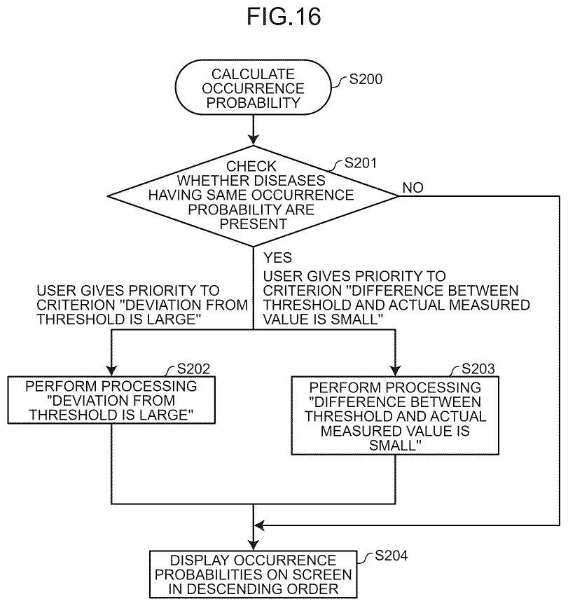

As illustrated in FIG. 15, it is considered that patterns of deviation include: a pattern in which a condition of occurrence probability is satisfied and the difference between a threshold and an actual measured value is large; and a pattern in which a condition of occurrence probability is not satisfied but the difference between a threshold and an actual measured value is small. Here, processing performed when occurrence probabilities are identical is described using FIG. 16.

FIG. 16 is a flowchart of the second modification illustrating a procedure of processing performed when occurrence probabilities are identical.

First, the occurrence probabilities of a plurality of diseases are calculated by the calculation function 152 (step S200). At this time, the display control function 153 checks whether or not a plurality of diseases has the same occurrence probability (step S201).

If a plurality of diseases does not have the same occurrence probability (No at step S201), the display control function 153 causes the occurrence probabilities of the diseases to be displayed in the display area 212 of the screen 200 in descending order of occurrence probability, as described above (step S204).

In contrast, if a plurality of diseases has the same occurrence probability (Yes at step S201), the display control function 153 checks setting information set by the user.

For example, it is assumed that, as the setting information, an instruction to give priority to a criterion “Deviation from threshold is large” is set in advance by the user. In this case, the display control function 153 performs the later-mentioned processing “Deviation from threshold is large” (step S202), followed by processing at step S204.

For example, it is assumed that, as the setting information, an instruction to give priority to a criterion “Difference between threshold and actual measured value is small” is set in advance by the user. In this case, the display control function 153 performs the later-mentioned processing “Difference between threshold and actual measured value is small” (step S203), followed by processing at step S204.

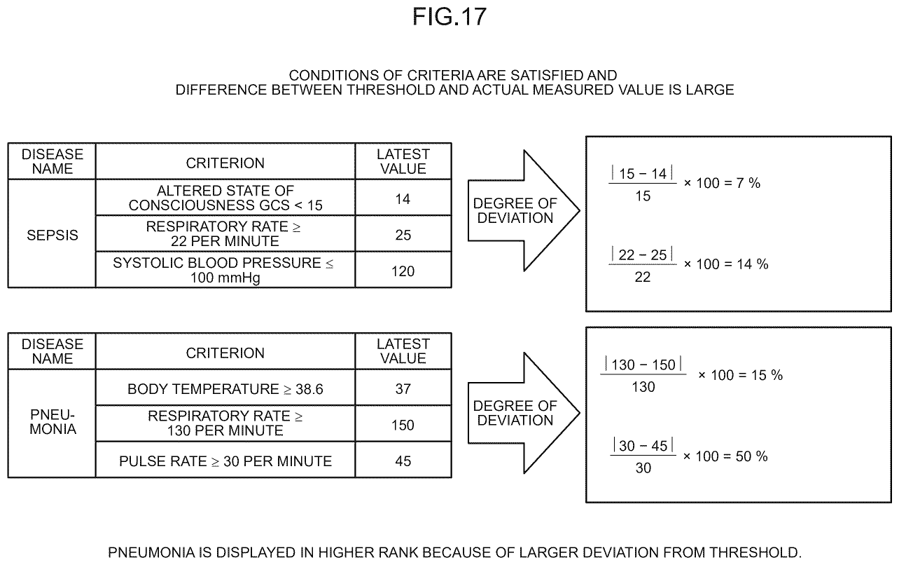

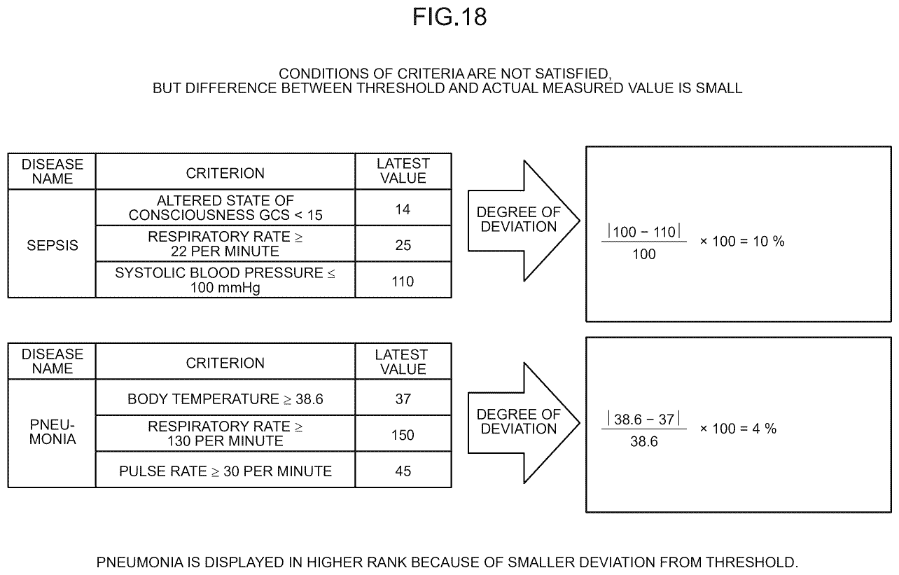

As the processing at step S202 and step S203, processing performed when the occurrence probabilities are identical will be described in detail by using FIG. 17 and FIG. 18.

FIG. 17 is a diagram illustrating processing performed when conditions of the criteria are satisfied and the difference between a threshold and an actual measured value is large, as the processing at step S202 in FIG. 16.

In the example illustrated in FIG. 17, it is assumed that a plurality of diseases having the same occurrence probability is “sepsis” and “pneumonia”.

Examples of the diagnostic items for “sepsis” include the altered state of consciousness, a respiratory rate, and systolic blood pressure. In the diagnostic item of the altered state of consciousness, whether GCS is 15 points or lower is diagnosed as the disturbance of consciousness of a patient; in the diagnostic item of respiratory rate, whether the respiratory rate of the patient is 22 or more per minute is diagnosed; and, in the diagnostic item of systolic blood pressure, whether the systolic blood pressure of the patient is 100 mmHg or lower is diagnosed. For example, it is assumed that, in the diagnostic item of the altered state of consciousness, the latest value of GCS is 14 points as a disturbance of consciousness of a patient; in the diagnostic category of respiratory rate, the latest value of respiratory rate of the patient is 25 per minute; and in the diagnostic item of systolic blood pressure, the latest value of systolic blood pressure of the patient is 120 mmHg. In this case, among diagnostic items for “sepsis”, the diagnostic items of the altered state of consciousness and the respiratory rate apply, and the occurrence probability of sepsis is 67[%].

For example, diagnostic items for “pneumonia” include body temperature, respiratory rate, and pulse rate. In the diagnostic item of the temperature, whether the body temperature of the patient is 38.6 or higher is diagnosed. In the diagnostic item of respiratory rate, whether the respiratory rate of the patient is 130 or higher per minute is diagnosed. In the diagnostic item of pulse rate, whether the pulse rate of the patient is 30 or more per minute is diagnosed. For example, in the diagnostic item of body temperature, the latest value of the body temperature of a patient is 37 degrees Celsius; in the diagnostic item of respiratory rate, the latest value of the respiratory rate of the patient is 150 per minute; and in the diagnostic item of pulse rate, the latest value of the pulse rate of the patient is 45 per minute. In this case, among the diagnostic items for “pneumonia”, the diagnostic items of respiratory rate and pulse rate apply, and the occurrence probability of pneumonia is 67[%].

Here, among the diagnostic items for “sepsis”, diagnostic items that satisfy the conditions of the criteria are the diagnostic items of the altered state of consciousness and respiratory rate, and the display control function 153 checks the deviation between a threshold and an actual measured value of the diagnostic items of the altered state of consciousness and respiratory rate. For example, in the diagnostic item of the altered state of consciousness, the deviation is (|15−14/15)×100=7%, and in the diagnostic item of respiratory rate, the deviation is (|22−25/22)×100=14%.

Among the diagnostic items for “pneumonia”, diagnostic items that satisfy the conditions of the criteria are the diagnostic items of respiratory rate and pulse rate, and the display control function 153 checks the deviation between a threshold and an actual measured value of the diagnostic items of respiratory rate and pulse rate. For example, in the diagnostic item of respiratory rate, the deviation is (|130−150|/130)×100=15%, and in the diagnostic item of pulse rate, the deviation is (|30−45|/30)×100=50%.

In this case, out of the diseases “sepsis” and “pneumonia” having the same occurrence probability, the disease “pneumonia” has a larger deviation from the threshold, and accordingly the display control function 153 displays the disease “pneumonia” in a higher rank than the disease “sepsis” in the display area 212. Here, since the diseases “pneumonia” and “sepsis” have the same occurrence probability, when the display control function 153 causes the diseases “pneumonia” and “sepsis” to be displayed in the display area 212, the graph of the disease “pneumonia” and the graph of the disease “sepsis” overlap each other in the latest occurrence probability in graph form as illustrated in FIG. 6, FIG. 10, FIG. 13, or the like. Therefore, in the present modification, when the diseases are displayed in descending order of the latest occurrence probability, “pneumonia” is displayed above “sepsis” in the legends of the graph, as described above. Alternatively, when there are diseases having the same occurrence probability, a plurality of diseases may be displayed in tabular form in the order of the degree of deviation, with an indication “The ranking is based on the degree of deviation”.

FIG. 18 is a diagram illustrating processing performed when the conditions of criteria are not satisfied but the difference between a threshold and an actual measured value is small, as the processing at step S203 in FIG. 16.

In the example illustrated in FIG. 18, like the example illustrated in FIG. 17, it is assumed that a plurality of diseases having the same occurrence probability is “sepsis” and “pneumonia”. The example illustrated in FIG. 18 is different from the example illustrated in FIG. 17 in that, in the diagnostic item of systolic blood pressure for “sepsis”, the latest value of the systolic blood pressure of a patient is assumed to be 110 mmHg.

Here, among the diagnostic items for “sepsis”, a diagnostic item that does not satisfy the condition of the criterion is the diagnostic item of systolic blood pressure, and the display control function 153 checks the deviation between a threshold and an actual measured value for the diagnostic item of systolic blood pressure. For example, in the diagnostic item of systolic blood pressure, the degree of deviation is (|100−110|/100)×100=10%.

Among the diagnostic items for “pneumonia”, a diagnostic item that does not satisfy the condition of the criterion is the diagnostic item of body temperature, and the display control function 153 checks the deviation between a threshold and an actual measured value for the diagnostic item of body temperature. For example, in the diagnostic item of body temperature, the deviation is (|38.6−37|/38.6)×100=4%.

In this case, out of the diseases “sepsis” and “pneumonia” having the same occurrence probability, the disease “pneumonia” has a smaller deviation from the threshold, and accordingly the display control function 153 displays the disease “pneumonia” in a higher rank than the disease “sepsis” in the display area 212. In the present modification, when the diseases are displayed in descending order of the latest occurrence probability, “pneumonia” is displayed above “sepsis” in the legends of the graph, as described above. Alternatively, when there are diseases having the same occurrence probability, a plurality of diseases may be displayed in tabular form in the order of the degree of deviation, with an indication “The ranking is based on the degree of deviation”.

Thus, in the second modification, the display control function 153 can precisely rank a plurality of diseases by considering the deviation between a threshold and an actual measured value (the difference between a threshold and an actual measured value) related to criteria used to calculate the occurrence probability.

Note that, in the second modification, there is a possibility that, even when compared by the degree of deviation, a plurality of diseases having the same occurrence probability has the same degree of deviation. For example, there is a possibility that the deviation of a diagnostic item for “sepsis” and the deviation of a diagnostic item for “pneumonia” are identical. In this case, in the second modification, weights are assigned in advance to each diagnostic item so that ranking can be determined precisely. For example, when the degree of deviation of the diagnostic items for “sepsis” is the same as the degree of deviation of the diagnostic items for “pneumonia” in FIG. 18, “0.8” is set as weight for the diagnostic item of systolic blood pressure for “sepsis” and “0.9” is set as weight for the diagnostic item of body temperature for “pneumonia”. In this case, out of the diseases “sepsis” and “pneumonia” having the same occurrence probability, the display control function 153 displays the disease “pneumonia” in a higher rank than the disease “sepsis” in the display area 212 because both the diseases have the same degree of deviation but more weight is assigned to the disease “pneumonia”.

Thus, in the second modification, the display control function 153 can precisely rank a plurality of diseases by considering the deviation between a threshold and an actual measured value (the difference between a threshold and an actual measured value) related to criteria used to calculate occurrence probability and assigning weight to each diagnostic item.

### Third Modification

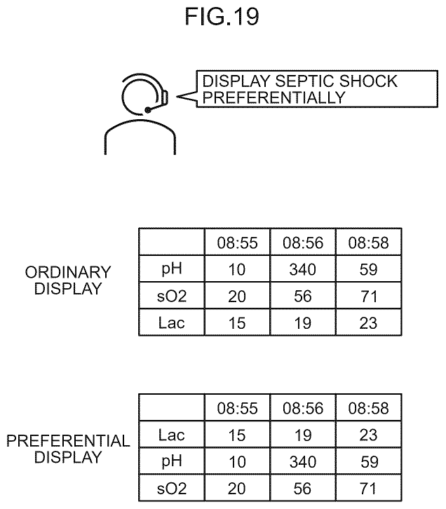

FIG. 19 is a diagram of a third modification illustrating a preferential display of parameters based on a disease.

For example, when a disease is determined by physician's judgment, a parameter of the disease is preferentially displayed. In the example illustrated in FIG. 19, it is assumed that, when the occurrence probabilities of a plurality of diseases are displayed in the display area 212 of the screen 200 in FIG. 5 and the user (physician) preferentially provides treatment for “septic shock” as an approach to disease, the user provides, by voice input, an instruction to determine the disease “Display septic shock preferentially”.

In this case, the display control function 153 switches the display so as to cause the display 120 to display information for the treatment of the disease “septic shock” preferentially, instead of ordinary information display. For example, when a hydrogen ion index “pH”, oxygen saturation “sO2”, and lactate “Lac” representing a lactic acid are displayed in this order in the ordinary display, lactate “Lac” is important for treatment for the disease “septic shock” and therefore lactate “Lac” is moved up to a higher rank as a preferential display.

Thus, in the third modification, when a disease is determined by physician's judgment, the display control function 153 causes the display 120 to display information for the treatment for the disease preferentially, whereby treatment can be assisted.

### Fourth Modification

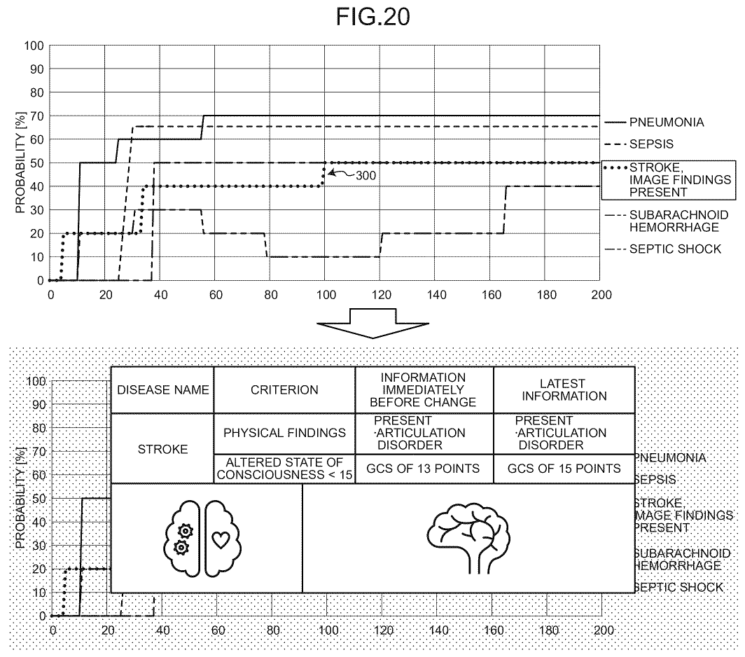

FIG. 20 is a diagram to explain a fourth modification. In FIG. 20, as the fourth modification, a display example is illustrated in which information about a trend change point (a point at which an index has changed) is displayed in a state in which the occurrence probabilities of a plurality of diseases are displayed in the display area 212 of the screen 200 in FIG. 5.

In the fourth modification, as for a disease whose occurrence probability [%] has changed with time, the display control function 153 causes the display 120 to display information before and after the change in the information concerning criteria used to calculate the occurrence probability [%].

Here, the information before the change is, for example, information immediately before the change. The information after the change is, for example, information immediately after the change or the latest information. The following describes a case in which the information immediately before and after the change and the latest information are displayed as information before the change in the information concerning the criteria.

In FIG. 20, the horizontal axis represents a time axis [seconds] and the vertical axis represent occurrence probability [%]. In the example illustrated in the upper figure of FIG. 20, the words “image findings present” are displayed in the display area 212 along with the disease “stroke”, as in FIG. 13, and the occurrence probability of the disease “stroke” changes from 40% to 50% after a lapse of 100 seconds. In this case, the display control function 153 causes the display 120 to display the information immediately before the change and the latest information about the criteria for stroke, as illustrated in the lower figure of FIG. 20. Note that, in the example illustrated in the lower figure of FIG. 20, images are also displayed in the display area 212.

In the example illustrated in the lower figure of FIG. 20, a value “GCS of 13 points” is displayed in the display area 212 as the information immediately before the change, and a value “GCS of 15 points” is displayed therein as the latest information. Furthermore, in the example illustrated in the lower figure of FIG. 20, the words “present, articulation disorder” are displayed in the display area 212 as information about physical findings immediately before the change and the latest physical findings.

The above description gives a case in which information before and after the change is automatically displayed when an index of occurrence (occurrence probability) changes, but the fourth modification may be a case in which information before and after the change is displayed on the request of the user.

In other words, when the user specifies a disease whose occurrence probability [%] has changed with the passage of time, the display control function 153 may cause the display 120 to display an index before the change and an index after the change or the latest index for criteria used to calculate the occurrence probability.

For example, in FIG. 20, it is assumed that, when 100 seconds have passed since the display of the occurrence probability of the disease “stroke”, the user specifies a change point 300 in the graph of stroke by using a mouse serving as an example of the input interface 110. In this case, the display control function 153 causes an image to be displayed in the display area 212 and also causes the display 120 to display the information immediately before the change and the latest information concerning the criteria for stroke.

By producing such display of the fourth modification, a physician can understand a factor in the change of occurrence probability.

### Fifth Modification

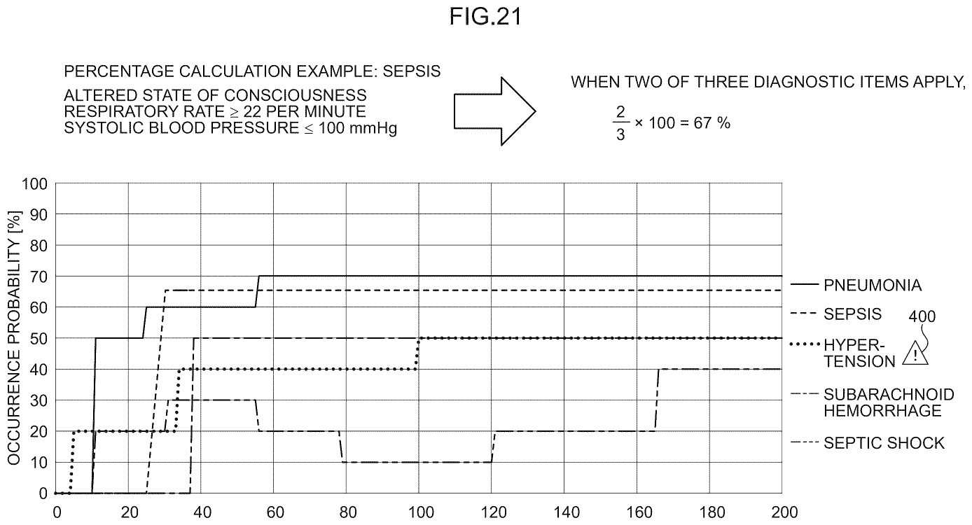

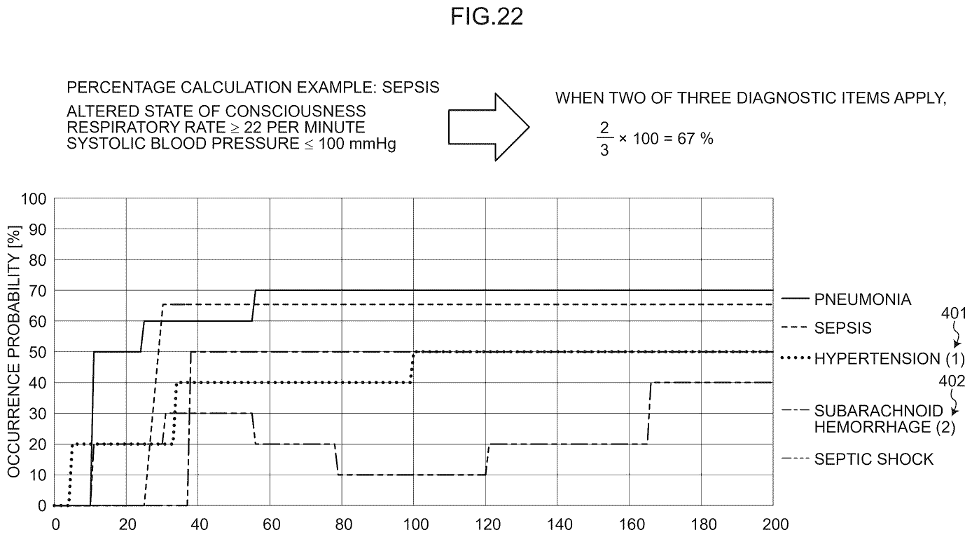

FIG. 21 is a diagram illustrating a display example in a fifth modification, and FIG. 22 is a diagram illustrating another display example in the fifth modification.

The display control function 153 causes the display 120 to display the occurrence probabilities of a plurality of diseases as well as information on the order of priority of treatment.

For example, as illustrated in FIG. 21, in the display area 212, the latest occurrence probabilities of the diseases “pneumonia”, “sepsis”, “hypertension”, “subarachnoid hemorrhage”, and “septic shock” decrease in that order. However, depending on a factor in the occurrence of a disease, the descending order of occurrence probability is not necessarily equal to the order of priority of treatment. The order of treatment is specified in advance in a hospital, for example.

For example, it is assumed that a disease to which treatment needs to be applied with first priority is “hypertension” and a disease to which treatment needs to be applied with second priority is “subarachnoid hemorrhage”. In this case, the display control function 153 associates information 400 (in FIG. 21, an exclamation mark) indicating that treatment needs to be applied with first priority, with the disease “hypertension” and causes the display 120 to display them.

Alternatively, as illustrated in FIG. 22, the display control function 153 associates a mark (“(1)” in FIG. 22) indicating the priority of treatment “first priority” as

information 401 about the order of priority of treatment with the disease “hypertension” and causes the display 120 to display the mark, and also associates a mark (“(2)” in FIG. 22) indicating the priority of treatment “second priority” as information 402 on the order of priority of treatment with the disease “subarachnoid hemorrhage” and causes the display 120 to display the mark.

Note that, in the fifth modification, an order determined in advance in a hospital may be changed in accordance with patient information (for example, past medical history). For example, when a patient has developed “subarachnoid hemorrhage” in the past, “subarachnoid hemorrhage” may be given “first priority” and “hypertension” may be given “second priority”.

Thus, in the fifth modification, the display control function 153 causes the display 120 to display the information about the order of priority of treatment, whereby efficient emergency treatment can be assisted.

Note that the functions of the information processing apparatus 100 in the information processing system 1 described above may be provided in a biological information collecting apparatus, for example.

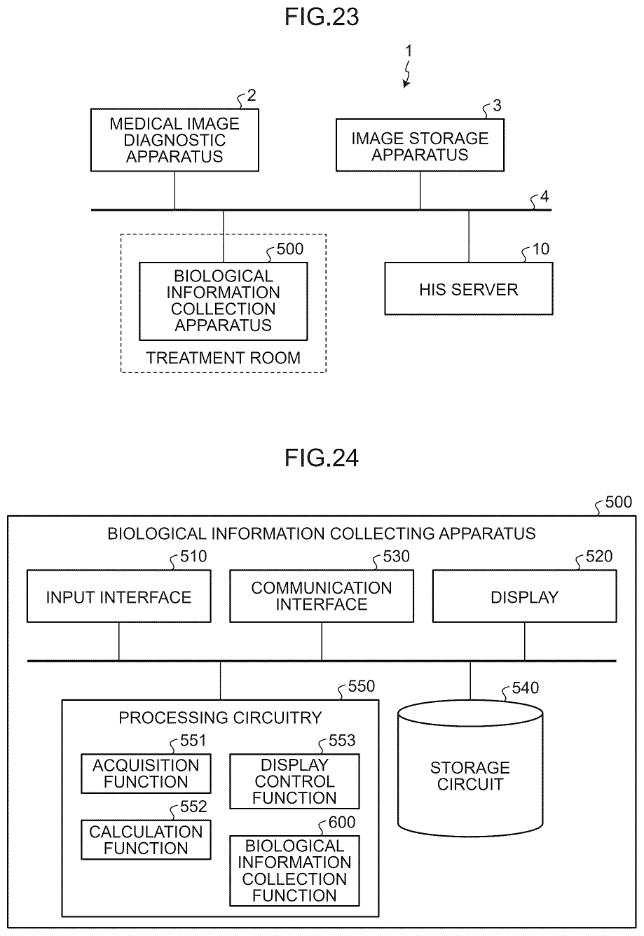

FIG. 23 is a diagram illustrating an example of the configuration of an information processing system including a biological information collecting apparatus 500. For example, the functions of the information processing apparatus 100 are implemented by the biological information collecting apparatus 500 illustrated in FIG. 24.

For example, the biological information collecting apparatus 500 is a device configured to collect patient's biological information from various biological information measuring devices. The biological information collecting apparatus 500 is provided in a treatment room in which emergency patients are treated, and the biological information collecting apparatus 500 mainly performs processing by using patient information acquired in the treatment room. Furthermore, the biological information collecting apparatus 500 can also apply various types of processing to medical information acquired from the HIS server 10, the medical image diagnostic apparatus 2, the image storage apparatus 3, and other devices.

FIG. 24 is a diagram illustrating an example of the configuration of the biological information collecting apparatus 500. As illustrated in FIG. 24, the biological information collecting apparatus 500 includes an input interface 510, a communication interface 530, a display 520, a storage circuit 540, and a processing circuitry 550. The input interface 510, the communication interface 530, the display 520, and the storage circuit 540 correspond to the input interface 110, the communication interface 130, the display 120, and the storage circuit 140 of the information processing apparatus 100 in FIG. 2, respectively.

The processing circuitry 550 controls the constituents of the biological information collecting apparatus 500. For example, the processing circuitry 550 implements an acquisition function 551, a calculation function 552, a display control function 553, and a biological information collection function 600.

The biological information collection function 600 collects patient's biological information and causes the display 520 to display the biological information. Examples of the biological information that can be collected by the biological information collection function 600 include heart rates, respiratory rates, blood pressure, electroencephalograms, electrocardiograms, and electromyograms, and pulse waves. The acquisition function 551, the calculation function 552, and the display control function 553 correspond to the acquisition function 151, the calculation function 152, and the display control function 153 of the information processing apparatus 100 in FIG. 2, respectively.

Note that the constituents of the devices illustrated in the present embodiment are only for conceptually illustrating the functions thereof and do not necessarily have to be physically configured as illustrated in the drawings. In other words, the specific forms of separation and integration of the devices are not limited to the illustrated forms, and all or part of each device can be functionally or physically separated and integrated in any unit in accordance with various loads, usage conditions, and the likes. Furthermore, all or any part of the processing functions performed by the devices can be implemented by a CPU and by a computer program analyzed and executed by the CPU or implemented as hardware by wired logic.

The processes described in the present embodiment may be implemented by executing a previously prepared computer program on a computer such as a personal computer or a workstation. The computer program can be distributed via a network such as the Internet. The computer program can also be recorded on a non-transitory computer readable medium, such as a hard disk, a flexible disk (FD), a CD-ROM, an MO, or a DVD, and executed by being read from the recording medium by a computer.

According to at least one of the embodiments described above, the efficiency of differentiation can be enhanced.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A biological information collecting apparatus, comprising:
processing circuitry configured to
acquire information about patient's condition,
calculate indexes related to occurrence of a plurality of diseases, based on the information acquired, and
causes a display to display the index related to occurrence of each of the diseases.

2. An information processing apparatus, comprising:
processing circuitry configured to
acquire information about patient's condition,
calculate indexes related to occurrence of a plurality of diseases, based on the information acquired, and
causes a display to display the index related to occurrence of each of the diseases.

3. The information processing apparatus according to claim 2, wherein
the processing circuitry
sequentially acquires the information about the patient's condition,
updates the indexes related to occurrence of the diseases, based on the information sequentially acquired, and
sequentially updates the index related to occurrence of each of the diseases, the index related to occurrence being displayed by the display.

4. The information processing apparatus according to claim 2, wherein the processing circuitry causes the display to display information about order of priority of treatment along with the index related to occurrence of each of the diseases.

5. The information processing apparatus according to claim 3, wherein the processing circuitry causes the display to display the index related to occurrence sequentially updated of each of the diseases on a time-series basis.

6. The information processing apparatus according to claim 5, wherein, for a disease whose index related to occurrence undergoes a change with time, the processing circuitry causes the display to display information about a criterion used to calculate the index related to occurrence, the information being obtained before and after the change.

7. The information processing apparatus according to claim 5, wherein, when a user specifies a disease whose index related to occurrence undergoes a change with time, the processing circuitry causes the display to display information about a criterion used to calculate the index related to occurrence, the information being obtained before and after the change.

8. The information processing apparatus according to claim 3, wherein the processing circuitry causes the display to display the index related to occurrence sequentially updated of each of the diseases in graph form on a time-series basis.

9. The information processing apparatus according to claim 2, wherein the processing circuitry causes the display to display information about a criterion used to calculate an index related to occurrence of a disease specified by user's input out of the diseases.

10. The information processing apparatus according to claim 2, wherein the processing circuitry causes an index related to occurrence of a disease satisfying a predetermined condition, out of the diseases, to be displayed.

11. The information processing apparatus according to claim 10, wherein the processing circuitry causes information about a disease not displayed by the display to be displayed on request of a user.

12. The information processing apparatus according to claim 2, wherein, when image-based findings are present in at least one of the diseases, the processing circuitry causes the display to display presence of the image-based findings in the disease.

13. The information processing apparatus according to claim 2, wherein, when priorities are assigned to the diseases, based on index related to occurrence, the processing circuitry adjusts the priorities, based on a difference between a threshold of a criterion used to calculate the index related to occurrence and an actual measured value.

14. An information processing method, comprising:
acquiring information about patient's condition;
calculating indexes related to occurrence of a plurality of diseases, based on the information acquired; and
causing a display to display the index related to occurrence of each of the diseases.

15. A non-transitory computer readable medium comprising instructions that cause a computer to execute:
acquiring information about patient's condition;
calculating indexes related to occurrence of a plurality of diseases, based on the information acquired; and
causing a display to display the index related to occurrence of each of the diseases.
